(19) United States
(12) Patent Application Publication
(10) Pub. No.: US 2008/0020453 A1
(43) Pub. Date: Jan. 24, 2008

(54) Analytical system based on porous material for highly parallel single cell detection

(76) Inventors: Thomas Ehben, Weisendorf (DE); Christian Zilch, Leipzig (DE)

Correspondence Address: HARNESS, DICKEY & PIERCE, P.L.C., P.O.BOX 8910, RESTON, VA, 20195, US

(21) Appl. No.: 11/878,088

(22) Filed: Jul. 20, 2007

(30) Foreign Application Priority Data
Jul. 21, 2006 (DE) 10 2006 033 875.8

(51) Int. Cl. C12M 1/33; C12M 1/34

(52) U.S. Cl. 435/287.2; 435/306.1

(57) ABSTRACT

At least one embodiment of the present invention relates to analytical systems based on porous material, for example silicon, for highly parallel single cell detection. At least one embodiment of the present invention relates in particular to porous silicon having a multiplicity of continuous channels and/or to the use thereof at least for cell separation, for cell lysis and purification of target molecules, for amplification of nucleic acid molecules or for detection of desired target molecules. At least one embodiment of the present invention also relates to an analytical method using porous silicon. Monoclonal antibodies for cell separation and immobilized capture molecules for cell lysis and purification are attached to the inside walls of the channels.

### PRIORITY STATEMENT

The present application hereby claims priority under 35 U.S.C. §119 on German patent application number DE 10 2006 033 875.8 filed Jul. 21, 2006, the entire contents of which is hereby incorporated herein by reference.

### FIELD

Embodiments of the present invention generally relate to analytical systems based on porous material, for example silicon, glass or plastic, for highly parallel single cell detection. Embodiments of the present invention may generally relate to porous material having a multiplicity of continuous channels and to the use thereof at least for cell separation, for cell lysis and purification of target molecules, for amplification of nucleic acid molecules and/or for detection of desired target molecules. Embodiments of the present invention also generally relate to analytical methods using porous material having a multiplicity of continuous channels.

### BACKGROUND

There is an increasing need in pharmaceutical research for methods and technologies for the rapid and accurate analysis of nucleic acids and proteins and other molecular target structures from biological samples. The development of rapid tests which can be carried out in parallel for genomic and proteomic analyses, pharmacokinetics and toxicity studies drives to a substantial extent the development of pharmaceutical and diagnostic products. In addition, there is an increased need for diagnostic systems for the highly parallel genomic and proteomic analysis of large cell populations and individual cells from a tissue assemblage, for example a tumor biopsy. Such systems help to save considerable costs and increase the throughput substantially by using automated systems operating in parallel. Clinical diagnostics and research into diseases as well as drug development increasingly make use of microarrays which have made it possible to decode disease-relevant modifications in cells and tissues.

It is possible to determine the concentration or the presence of biomolecules (e.g. DNA, proteins) in biological samples with the aid of biochips. Specific capture molecules such as oligonucleotides, antibodies or haptens are located at distinct sites (spots) on the surface of suitable supports (such as, for example, glass, Plexiglas, silicon) in microarrays. The advantage of currently developed microarrays over conventional analytical methods (in particular homogeneous assay systems) lies especially in the fact that many different biological problems can be addressed in a single experiment. Conventional microarrays are in general suitable for analyzing a multiplicity of different biological parameters in a sample. Thus, for example, the expression profiles of many genes or proteins, a large number of SNPs or gene mutations or the presence of genetic sequences, expressed mRNA or proteins can be detected simultaneously.

As a rule, after proteins or nucleic acids have been extracted from the cells of a sample, the target molecules to be detected are bound to the specific capture molecules immobilized to a support and detected by way of various types of markers. This requires the particular marker to couple to the target molecule to be detected. Optical as well as magnetic, electric or gravimetric methods are employed for detecting the particular marker. The sensitivity of these methods is very different. However, it can be generally stated that a sample must contain high concentrations of target molecules for the latter to be detected after binding to capture molecules. Most of the methods based on microarrays therefore require a large amount of sample starting material in order to ensure said sufficiently high concentration of target molecules. Depending on the sample, several 100 μl or μg of body fluid (e.g. blood) or tissue material (biopsy or swab) are required here to obtain the necessary number of cells or biological particles containing the target molecules. In some cases, however, there is not enough cell material or the number of (diseased) cells to be detected in the whole sample material is so low that the target molecules must be amplified prior to detection on a microarray.

Nucleic acid analysis employs a plurality of amplification methods such as, for example, polymerase chain reaction (PCR), ligase chain reaction (LCR) or rolling cycle amplification. Disadvantageously, however, all of these methods are limited with respect to the number of samples that can be analyzed simultaneously. Although it is possible to carry out separate amplification reactions, for example in the cavities of a microtiter plate, only a single sample can be measured in the subsequent hybridization on a microarray. Although many different biological parameters of each individual sample can be determined simultaneously, the current microarray designs do not allow any differentiated measurement of biological parameters from individual cells or cells of different pooled samples. This can be attributed to the fact that the supports are plane surfaces onto which the capture molecules are spotted or synthesized. Each sample (such as, for example, DNA or RNA) wets the entire surface of the microarray so that all spots come into contact with the particular sample. This fact results in a low sample throughput in microarray experiments. In contrast, a few biological parameters of a large number of samples are determined in laboratory medicine, in particular in mass screening. Currently, however, no microarray-based systems are in use which allow multiple parameters to be determined, with high sample throughput at the same time.

Another fundamental problem is the heterogenicity of some of the sample materials used. For example, a tumor biopsy consists of normal cells and degenerated cells of different degrees of malignancy. Bacteriological samples are usually mixtures of various pathogens of different pathogenicity. In contrast, viral samples comprise latent, in addition to active, viral gene material in infected cells or a body fluid (urine, blood plasma, lymph). The desired cells or biological particles are often in a minority compared to other “normal” cells or non-pathogens occurring in the sample. It is especially difficult to detect the cells or pathogens associated with a disease against the background of “normal” cells or non-relevant bacteria or viruses (“needle-in-a-haystack problem”). Owing to the heterogenicity of the cell types present in a sample, even the specific amplification methods such as, for example, PCR usually produce a result which represents a mixed culture or heterogeneous sample. This is the case in particular if the gene sequences in the various cell populations are similar. For the above reasons, there is a need for cost-effective methods which enable multiple biological parameters from heterogeneous cell populations to be determined with high throughput and which allow an accurate statistical analysis of the genomic or proteomic information.

In order to address the above-described problems, the heterogeneous cell populations or biological particles are currently fractionated with the aid of selection techniques such as fluorescence-activated cell sorting (FACS) or functionalized magnetic beads. If the sample material comprises histological sections, laser microdissection (LMD) methods are employed to extract special regions within a tissue section in order to subject the cells thus obtained to subsequent further analytical methods such as immunostaining or molecular-biological techniques. Essentially, however, only mixtures of selected cell types or biological particles are generated. An analysis based on a single cell is extraordinarily difficult. This fact proves to be a particular disadvantage in the following fields of application:

a) extensive population-genetic studies require highly paralleled processing of biochemical processes, starting with cell sorting and disruption methods, amplification of the desired genetic information, detection and quantification of the target sequences. Automating the various requisite steps requires a complicated laboratory infrastructure and robotics. Currently, the steps are carried out on “microtiter plates” which are commercially available in different formats. The common formats range from 96, 384 to 1536 well plates, limiting the number of samples processable at the same time. Moreover, the various processes necessitate constant changing of buffer fluids in each individual well, requiring precise robot control. The time required for thousands of patient samples is from a few days up to several weeks, depending on plate format, machine throughput and biological problem. This means, apart from the enormous amount of time needed, high costs in material and personnel.

b) the mapping of tumors involves preparing histological tissue sections which are treated with dyes enabling various types of cells and tissues to be distinguished. More recently, monoclonal antibodies have been used to identify special intra- and extracellular structures that characterize tumor cells. In this way it is possible to determine the degree of differentiation and the metastasizing potential of degenerated cells and the boundary to healthy cells within the affected organ. Recently, genetic analytical methods have increasingly gained importance, since they can deliver additional information about the tumor cells and their sensitivity to chemotherapeutics. For this purpose, histologically conspicuous regions in sections are removed with the aid of laser dissection methods and subjected to a genetic analysis. This usually involves generating gene expression profiles which allow a statement regarding the metabolism of tumor cells, or mutation profiles which provide the physician in charge with information about the affected genes, thus enabling the tumor to be categorized. The problem with the methods currently in use is the fact that processing of histological sections is very complicated and requires highly qualified personnel in order to achieve a meaningful result. The relevant regions in tumors are often not successfully identified in histological sections, and this may lead to false statements about the degree of differentiation and metastasizing or the sensitivity of said tumor to chemotherapeutics.

c) determining the number of particles and infected host cells is extraordinarily important in the diagnosis and monitoring of viral diseases. The number of latently infected cells and the dynamics of virus production play an important part, in particular in the context of progressive pathogenesis. Therefore, a number of viral assays have been developed which make it possible to establish the number of viral copies (viral load) in a defined amount of body fluid. This information provides important evidence regarding the course of a viral disease or a success of an antiviral therapy. However, a problem is the distinction between latently infected but inactive host cells and those which actively produce viral particles. Although the currently used methods of determining viral load enable viral copies (RNA or DNA) to be approximately determined in a defined amount of body fluid, it has not been possible so far to determine the number of cells in a cell population which are actually infected. It is furthermore extraordinarily difficult to infer the number of infected cells from the number of amplified viral copies. This would require analyzing each individual cell of a population. Although single cell determination is possible by using sensitive amplification methods currently in use, meaningful statistics often prove impractical due to the tiny number of infected cells in a population of noninfected cells. A number of selection methods currently developed make use of novel microfluidic concepts. For example, the Fraunhofer-Institut für Biomedizinische Technik (IBMT) has developed “microcapillary biochips” which allow analyzing of single cells and tissue samples for functional proteomics and biomonitoring. The systems enable automated screening of toxicological substances on minute cell and tissue models by using impedance spectroscopy. Possible applications arise in pharmaceutical drug screening, in quality control in food technology or in environmental technology. However, the throughput is limited to a few hundred samples per day.

IZKF Leipzig, Germany, have developed a new platform technology which enables a miniaturized laboratory for cell research to be established on a microchip. There, like in a marshalling yard, individual cells can be gently kept, sorted, characterized and treated in an electric cage. A similar technology has been developed at CEA in Grenoble, France, within the framework of the MeDICS project. The miniaturized cell sorters can be used to manipulate cells without mechanical contact with the aid of dielectrophoretic fields. Although both technologies make possible in principle a subsequent genomic and proteomic analysis of single cells, said analysis is currently still in the experimental stage and has not yet been integrated in diagnostic systems. Owing to the serial sorting, sample throughput is likewise limited.

Although very sensitive amplification methods meanwhile allow the sorted cells to be analyzed based on single cells, this requires in each case a separate assay. Single cell systems for high throughput analysis are supplied by various companies, among them Evotec OAI, Cybio, Tecan, Beckman Coulter, Guava Technologies, etc. However, all methods are limited to a throughput of a few thousand samples or cells per run, i.e. it is currently not possible to subject several million cells to single cell analysis in parallel and within a short period of time. Only a small number of single cell analyses is successful using the methods currently in use.

Histological sections of tumor material, complex population studies or pathogen differentiation in mixed cultures, however, usually comprise several million different cells in order to obtain meaningful statistical information. The abovementioned methods are too complicated, tedious, expensive or technically impracticable for these problems, in order to carry out simultaneously a molecular analysis based on single cells for a high number of various cell types.

U.S. Pat. No. 5,843,767 discloses a flow-through chip having a multiplicity of discrete channels which extend from a first surface to an opposite second surface. The channels have a diameter of from about 0.033 μm to about 10 μm. A first binding reagent for binding a target molecule is immobilized to the walls of the channels. The binding reagent is suitable for carrying out an analytical task of generating profiles of cell populations. A PCR reaction is proposed for detection, with the flow-through chip lacking a heating element necessary therefor.

DE 101 42 691 discloses an analytical method using a macroporous substrate which has opposite first and second surfaces, wherein a multiplicity of discrete pores which have a diameter of from 500 nm to 100 μm and which extend through the substrate from the first to the second surface are arranged over a surface region. An analyte is immobilized location-specifically by at least one capture molecule to the inside wall surfaces of each pore. This is followed by measuring a changeable light transmission property of the pore as a function of a binding reaction between capture molecule and analyte. The analyte is, for example, a single cell. For this purpose, light is coupled into the pore and detected by a CCD array at the opposite side. Naturally, without a step of amplification of the analyte, said change in the light transmission property can be detected only with complex equipment and a moderate signal-to-noise ratio.

Another system for using a flow-through chip with a capillary cassette is disclosed by DE 200 22 783 U1. Said capillary cassette has a substrate and a multiplicity of capillaries which extend through the substrate and have an open end each on opposite sides of said substrate. In order to amplify nucleic acid by means of a PCR reaction, the open ends are sealed with a membrane. The reaction is carried out by introducing the cassette to a thermocycler. A PCR reaction can be carried out by means of an air stream heated by a heating element in the thermocycler. A particular disadvantage of this system is the fact that the reaction products must be distributed from the capillaries for detection. Furthermore, an analyte bound to the capillary produces a significantly reduced signal during detection compared with an unbound analyte under identical conditions.

### SUMMARY

In at least one embodiment of the present invention, a device may be provided which can be used to carry out the above-described analyses in a more inexpensive and simpler manner and with higher throughput.

In at least one embodiment, this may be achieved by at least one of a flow-through chip made of a porous material, a use of a flow-through chip made of porous material and/or also an analytical method.

According to at least one embodiment of the invention, preference is given to modifying the already established flow-through-chip solution based on porous silicon, which has previously been used for gene expression studies, in such a way that completely new fields of application such as highly paralleled single cell analysis can be opened up. The porous material is used in the novel methods preferably as a nanotiter plate or nanotiter chip in which each individual, continuous channel can be used for cell separation, as a lysis channel, amplification channel and detection channel. Apart from highly paralleled processing of many samples, an analysis of individual cells is also possible based on genomics and potentially also on proteomics.

The porous material may be any material which can have a multiplicity of continuous microchannels with a diameter in the order of magnitude range from 0.1 to 100 μm. The microchannels are preferably essentially parallel to one another. The material may therefore be both porous silicon, as described in the example embodiment hereinbelow, and porous glass or porous plastic. Suitable porous glass is prepared, for example, by repeatedly heating a bundle of glass fibers elongating said bundle and compressing it. Alternatively, it is also possible to oxidize a porous silicon chip to give glass, for example by prolonged heating in a suitable atmosphere.

A suitable porous plastic material is, for example, epoxide resin, since it has very good binding properties for biological molecules. The porous structure may be prepared by various methods, for example by additive methods from the “rapid manufacturing” field (microstereolithography), by micromold technique which involves preparing a suitable casting mold by methods of the microsystem technique, or by abrasive processing methods such as SU-8-.

It has previously not been possible to study a multiplicity of samples simultaneously by using systems such as, for example, the flow-through system developed by Infineon and Metrigenix. In addition, sample preparation still had to be carried out separately. Only after the nucleic acids and their label have been isolated, are all other steps of hybridization and subsequent detection controlled with the aid of the 3D or 4D fluidic systems. Advantageously, however, at least one embodiment of the invention enables cell binding as well as lysis, nucleic acid isolation, amplification and detection to be carried out inside each individual channel. For this purpose, antibodies directed to a desired cell or to the target protein are attached to the wall of each individual channel. For genomic applications in nucleic acid diagnostics, single-stranded oligonucleotides which are capable as capture molecules to bind gene sequences in each individual cell are fixed to the inside walls of the channels. Preference is given to controlling the microfluidics by using a modified 3D flow-through system developed by Infineon.

### BRIEF DESCRIPTION OF THE DRAWINGS

Example embodiments of the invention will now be described with reference to the accompanying drawings in which

FIG. 1 depicts an example embodiment of porous material in the form of a flow-through chip which, at the same time, has the form of a nanotiter plate;

FIG. 2 depicts an arrangement of the flow-through chip according to FIG. 1 in an analytical system;

FIG. 3 depicts a histological section arranged on the flow-through chip according to FIG. 1;

FIG. 4 depicts an example of a virological application using the flow-through chip according to FIG. 1;

FIG. 5 depicts a cross-sectional view of the flow-through chip according to FIG. 1, wherein statistically a single cell is bound to an antibody in the channel;

FIG. 6 depicts an enlarged cross-sectional view of FIG. 5, wherein a DNA of the cell is released; and

FIG. 7 depicts an enlarged cross-sectional view of FIG. 5, wherein a PCR and detection of desired molecules are carried out.

The example embodiments of the present invention are described below with reference to the drawings.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described. Like numbers refer to like elements throughout. As used herein, the terms “and/or” and “at least one of” include any and all combinations of one or more of the associated listed items.

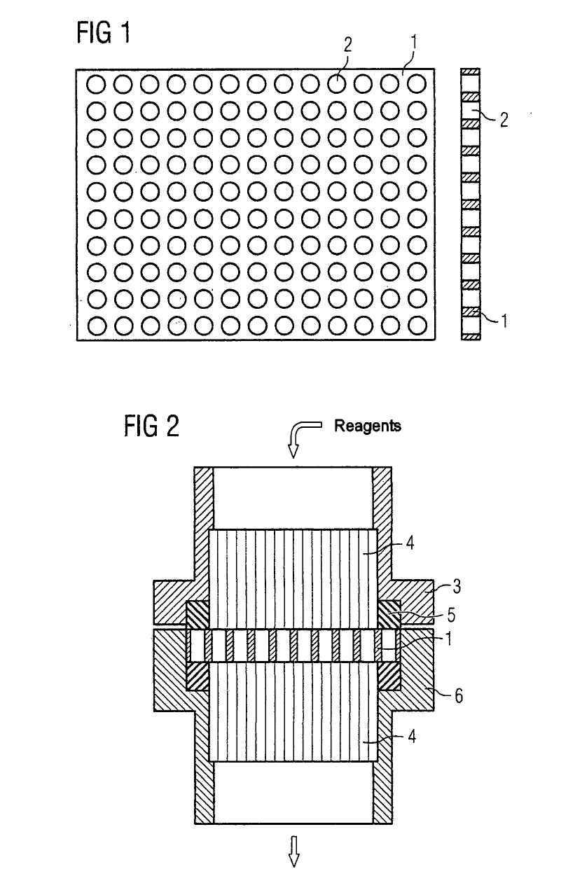

FIG. 1 depicts an example embodiment of porous material in the form of a flow-through chip 1 made of silicon, which, at the same time, has the form of a nanotiter plate, wherein the channels 2, however, completely penetrate the flow-through chip 1.

The porous silicon employed in said flow-through chip 1 includes a single crystal plate of a few hundred micrometers in thickness, into which a few hundred thousand identical channels 2 per square centimeter of surface have been etched. The channels 2 completely run through the plate of the flow-through chip 1, i.e. they extend from the top to the bottom. The diameter of the channels 2 may be chosen by choosing etching conditions to be between approx. 0.1 and 100 μm. The conditions must be chosen so as to provide space therein for a single eukaryotic or prokaryotic cell 13, as is indicated, for example, in FIG. 5. Two other important requirements must be met: 1) the channels 2 must be closable from the top and bottom of the flow-through chip 1 in a variable manner but not independently of one another, and 2) enable liquid reagents to pass through.

FIG. 2 depicts such an arrangement of the flow-through chip 1 according to FIG. 1 in an analytical system.

The closability is preferably implemented by way of a sealing sponge 4 and optionally by way of an upper and a lower movable cover plate 3, 6 made of polymeric material or membranes. The sealing sponge 4 seals under pressure to prevent in this way a longitudinal flow and may be compressed, for example, by the cover plates 3, 6 until sealing is achieved. Furthermore, the cover plates 3, 6 clamp the flow-through chip 1 with a seal 5 arranged between them. Optionally, electrical contacts and conductors may be applied to the seal 5 which, as a result, then serves, with voltage applied, as a heating element for heating the flow-through chip 1. A sealing sponge 4 is arranged downstream and upstream of the flow-through chip 1.

The passing through of various reagents is ensured by way of a flow chamber in the cover plates 3, 6, into which the reagents are pumped from the particular reservoirs (not shown).

Spatially separated biological reactions occur in each of the channels 2. Depending on the sample material (cell suspension or tissue section), the processing method must be chosen so that the cells are thinned out and distributed to the various channels 2 of the porous silicon 1. An example embodiment of an analytical method for single cell analysis is described below with reference to the following figures.

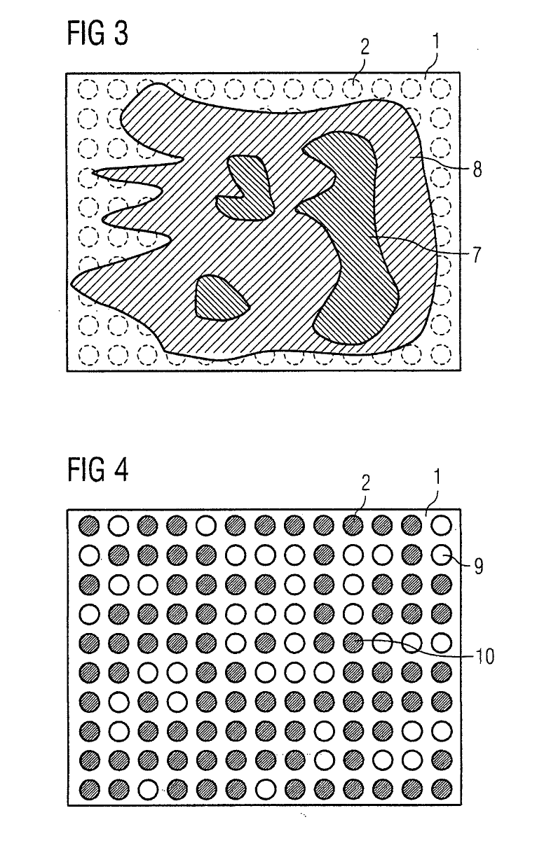

FIG. 3 depicts a histological section 8 which is arranged on the flow-through chip 1. The histological section contains tumor cells 7 which are to be identified.

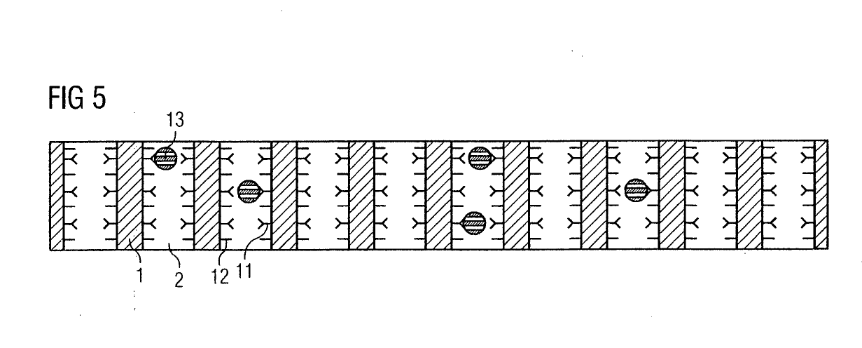

FIG. 4 alternatively depicts an example of a virological application using the flow-through chip 1. The empty channels 2 depicted contain healthy cells 9 and the solid channels 2 depicted contain latently infected cells 10.

The next step comprises separating cells. FIG. 5 depicts a cross sectional view of the flow-through chip 1, wherein a cell 13 is bound to an antibody 11 in the channel 2.

This involves firstly, for example, fixing chemically the histological section 8 to the surface of the porous silicon chip 1. After incubation in a suitable lysis reagent which dissolves the tissue assemblage (e.g. trypsinization or use of a different suitable protease), the cells 13 are thinned out and mechanically pressed into the channels 2, preferably with the aid of a die which acts on the tissue.

If however cell suspensions are used, then the monoclonal antibodies 11 coupled to the inside wall of the channels 2 ensure that, after pumping in the suspension through the porous silicon 1, statistically in each case a single cell 13 per channel 2 is bound. To this end, a part of each individual channel 2 is functionalized with active chemical groups (tosyl, amino, epoxy, thiol, etc.) and spotted with specific monoclonal antibodies 11 so as to cover the inside walls of the channels 2 with antibodies 11. The latter serve to bind specific target cells, for example lymphocytes such as CD4 or CD8 T helper cells, or cytotoxic cells. In this way it is possible to separate from one another and process further different cell populations from a mixture.

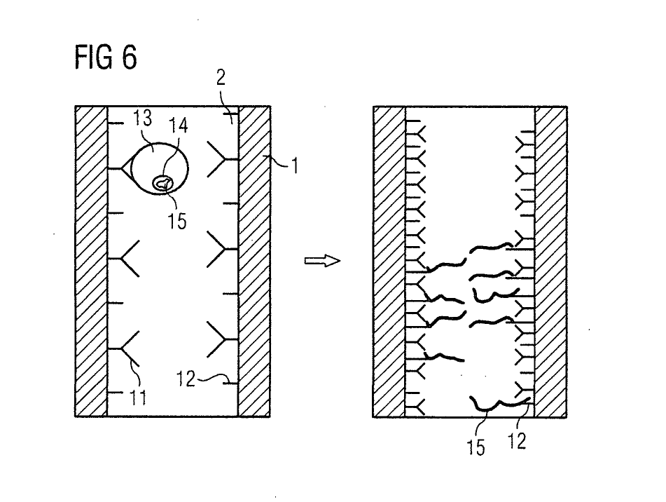

In the next step, cells may be lysed and the target molecules purified, as depicted in FIG. 6. FIG. 6 depicts an enlarged cross-sectional view of FIG. 5, wherein a DNA 15 of the cell 13 is released. The reference number 14 indicates a nucleus of the cell 13.

The cells 13 present in the channels 2 are then lysed chemically, biologically or thermally inside each channel 2. The released proteins or nucleic acids are bound by capture molecules 12 such as antibodies or oligonucleotides immobilized to the inside wall of the channels 2. In the case of a genomic assay, for example, lysis reagents which cause the RNA and DNA 15 to be released are passed through the channels 2. The genetic information may be bound with the aid of silanes or specific oligonucleotides 12 bound to the channels 2. In the case of proteomic assays, for example, the target proteins are bound by immobilized specific antibodies 12 to the inside wall of the channels 2. This is followed by direct or indirect identification by way of said detection or, if required, an amplification inserted into the process. Said amplification may be required in particular for an analysis of nucleic acid molecules 15, but there are also nucleic acid assays conceivable in which biological material is generated to such an extent that an amplification can be dispensed with.

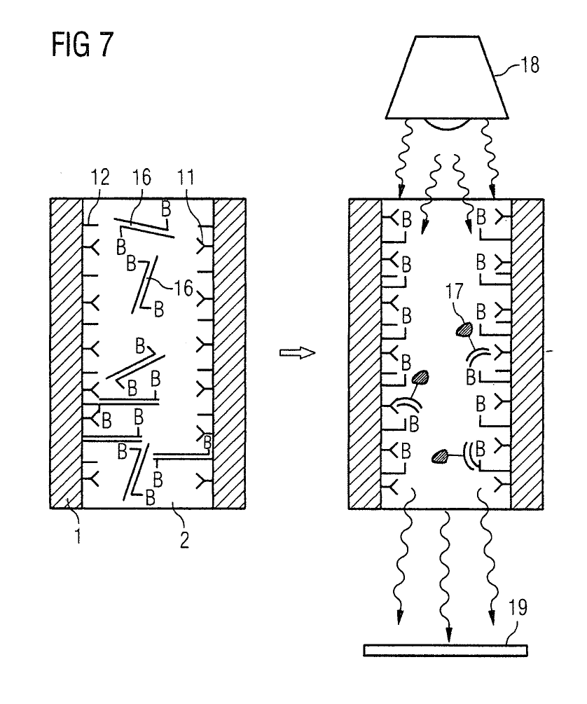

The amplification of nucleic acid molecules is depicted in the left-hand part of FIG. 7. FIG. 7 depicts an enlarged cross-sectional view of FIG. 5, wherein a PCR (polymerase chain reaction) is carried out for amplifying desired molecules 15.

Owing to the excellent heat conducting properties of the porous silicon 1, it is possible to carry out a temperature-controlled PCR in the individual channels 2. This is done, for example, by thermal coupling of a Peltier element (not shown) to the porous silicon chip 1 and/or by electrical heating due to the resistance of the material (e.g. semiconductor material). Optionally, it is also possible to heat by heating the seal 5 if the latter, as described with reference to FIG. 1, is conductive and provided with electrical contacts. Prior to this, the necessary PCR reagents are pumped into the chambers. Preferably single-stranded oligonucleotides which are labeled, for example, with biotin 16 and which have been attached to the inside walls of the channels 2 beforehand may be used for a solid phase PCR or primer extension. It must be ensured that the reagents do not escape due to diffusion during amplification of the target molecules. This may be achieved, for example, by sealing or covering the channels 2 on both sides with the aid of membranes or cover plates 3, 6, after pumping in the required reagents, as depicted in FIG. 2.

As an alternative which, however, is more complicated manually, the porous silicon chip 1 may be fixed, sandwich-like, between two Peltier elements provided with thin seals made of heat-conductive material (not shown). In this way, a PCR may be carried out inside the channels 2 of the porous silicon chip 1 in a manner similar to that in a microtiter plate but with very much less sample material (usually from a single cell 13 per channel 2) and in a substantially more paralleled manner (several hundred thousand separate reactions per square centimeter of silicon).

After cell lysis or after the optional amplification, the desired target molecules are detected, as depicted in the right-hand part of FIG. 7. As with the previously implemented applications, desired target molecules can be detected with the aid of optical detection methods (fluorescence or bioluminescence). In the arrangement in the right-hand part of FIG. 7, the channel 2 is illuminated by a light source 18. A detector 19, preferably a CCD chip or a CMOS camera, is arranged at the other end of the channel 2.

Preferably, immobilized specific capture molecules 17 which can interact with the desired target molecules are located on the inside walls of the channels 2 of the porous silicon chip 1. In the case of proteomic applications, antibodies or haptens are suitable for this. In the case of genomic applications, said capture molecules are oligonucleotides which hybridize with complementary sequences of the target molecules. The detection of specific mutations in a sequence section (SNPs) requires, depending on the sequence treatment, a stringency which can be adjusted by way of the buffer conditions and the temperature.

Following the attachment or hybridization process, the unbound labeled molecules must be washed out of the channels by pumping through buffer. Subsequently, the remaining markers bound via the target molecules with the capture molecules 17 are quantified by way of optical methods, for example by putting the silicon-chip 1 in an optical scanner. Similarly to the bioluminescence methods previously developed by Infineon and Metrigenix, enzymic amplification may also be employed for luminescent agents. Alternatively it is also possible here to use conventional magnetic, electric or other methods for detection of the target molecules. It is furthermore possible to establish a melting curve, i.e. a defined temperature range can be covered, within which the degree of hybridization can be determined dynamically.

At least one embodiment of the present invention is advantageous in the following applications:

Millions of different individual cells from different samples such as tumor tissue, mixed populations from blood samples or other body fluids, may be characterized and analyzed in a highly parallel manner at the genomic or proteomic level. The system may be employed in the fully integrated analysis of DNA and proteins. At least one embodiment of the method is therefore suitable in particular for the following applications:

gene expression profiles on single cell basis

identification of mutated or abnormal cells (cancer cells) in a complex mixture of cells

analysis of heterogeneous mixtures of cells

analysis of pooled samples (population genetics)

high throughput drug screening for identification of suitable candidate substances by way of conventional fluorescence or luminescence readout or confocal single cell or molecule detection

use of the flow-through chip for mass-spectrometric studies. To this end, primer extension is carried out on the solid phase inside the individual channels.

Compared with conventional analytical systems, at least one embodiment of the present invention is distinguished by the following advantageous properties:

Integration of many different processes from cell separation, nucleic acid preparation, amplification to molecular detection will be possible. Currently, only hybridization and the luminescence reaction are carried out in the channels of the chip.

It will be possible to study individual cells in each channel separately. In this way mutations and expression profiles of genes can be assigned to the particular single cell. For this purpose, for example, specific antibodies enabling cell adhesion could be placed in the channels.

The novel biochips made of porous silicon can be employed in mass spectrometry by introducing the required matrices into the individual chambers. In this way it is possible to analyze a substantially higher number of samples/genetic parameters than is the case with current MALDI methods (“matrix assisted laser dissociation and ionisation”).

The workflow is considerably simplified compared with systems common in laboratories, since complicated robotics are not required. Using the technology for studying histological sections no longer requires any complicated laser disection. Histological stainings and the subsequent genetic study of all regions of the entire histological section are possible on a single chip (with appropriate dimensions).

The application in viral load assays can provide information about the number of latently infected and actively virus-producing host cells. It is thus possible to determine, whether a certain number of viral copies can be attributed to a few active cells or to many latently infected cells.

The assay time is significantly reduced due to the enormous potential for parallel implementation. The parallel implementation of genomic assays, as it is currently driven forward by using microtiter plates with higher and higher densities, is multiplied again massively by using the porous silicon. It will be theoretically possible to carry out several million separate reactions on a few square centimeters of chip surface, thereby making better statistical evaluations of mass screenings possible.

The costs for laboratory robotics and in particular for the reagents are significantly reduced, since firstly the apparatus become smaller and secondly the required reagent volumes can be significantly reduced.

The method of at least one embodiment of the invention described herein has multiple steps for cell separation, for cell lysis and purification of target molecules, for amplification of nucleic acid molecules and for detection of desired target molecules. However, at least one embodiment of the present invention is not limited to the combination of the individual steps that is described herein. Depending on the application, particular steps may be omitted or further steps can be added.

The present invention is not limited to the illustrated embodiments but the scope of the invention, which is defined by the enclosed claims, likewise comprises modifications.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A flow-through chip made of a porous material and including a multiplicity of continuous channels, a diameter of the channels being between 0.1 and 100 μm so that there is space for a single eukaryotic cell and wherein the channels are closeable from a top and bottom of the flow-through chip in a variable manner, but not independently of one another, by movable cover plates, made of at least one of polymeric material and membranes, including a seal arranged between the plates which, with a voltage being applied, serves as a heating element for heating the channels to carry out a polymerase chain reaction, and wherein liquid reagents are passable through the channels, and including at least one of:
monoclonal antibodies, on inside walls of the channels, to separate statistically one desired cell per channel; and
immobilized capture molecules, on inside walls of the channels, to bind at least one of desired cells, desired target molecules, desired target proteins and desired gene sequences.

2. The flow-through chip as claimed in claim 1, wherein the porous material is designed as at least one of a nanotiter plate and a nanotiter chip.

3. The flow-through chip as claimed in claim 1, wherein the porous material is made from at least one of porous glass and porous plastic.

4. The flow-through chip as claimed in claim 1, wherein the porous material is made from porous silicon.

5. The flow-through chip as claimed in claim 4, wherein the porous silicon is in the form of a single crystal plate into which the channels are etched in a continuous manner.

6. A method, comprising:
using a flow-through chip made of porous material, as claimed in claim 1, for at least one of cell separation, cell lysis and purification of target molecules, amplification of nucleic acid molecules and detection of desired target molecules.

7. An analytical method using a flow-through chip made of porous material including a multiplicity of channels with a diameter of between 0.1 and 100 μm, comprising at least one of:
a step for cell separation in the channels;
a step for cell lysis and purification of target molecules in the channels;
a step for amplification of nucleic acid molecules in the channels; and
a step for detection of desired target molecules in the channels.

8. The analytical method as claimed in claim 7, wherein at least one of:
the step for cell separation involves applying a histological section to the surface of the porous material and thinning out the cells of the histological section for statistically one cell per channel to be bound;
the step for cell lysis and purification of the target molecules involves arresting the cells present in the channels by way of immobilized capture molecules inside each channel and at least one of lysing the cells biologically, lysing the cells chemically by way of lysis reagents, lysing the cells thermally, lysing the cells by way of ultrasound and lysing the cells mechanically/physically;
the step for amplification of nucleic acid molecules involves heating the channels by way of a heating element to carry out a polymerase chain reaction; and
the step for detection of desired target molecules involves specific capture molecules immobilized to inside walls of the channels interacting with the desired target molecules, subsequently washing unbound labeled molecules out of the channels by pumping through buffer, and then quantifying remaining markers bound via the target molecules with the capture molecules by way of at least one of optical, magnetic, electrochemical and radioactive methods.

9. The flow-through chip as claimed in claim 2, wherein the porous material is made from at least one of porous glass and porous plastic.

10. The flow-through chip as claimed in claim 2, wherein the porous material is made from porous silicon.

11. The flow-through chip as claimed in claim 10, wherein the porous silicon is in the form of a single crystal plate into which the channels are etched in a continuous manner.

12. The flow-through chip as claimed in claim 3, wherein the porous material is made from porous silicon.

13. The flow-through chip as claimed in claim 12, wherein the porous silicon is in the form of a single crystal plate into which the channels are etched in a continuous manner.
